(19) United States
(12) Patent Application Publication
(10) Pub. No.: US 2001/0047013 A1
(43) Pub. Date: Nov. 29, 2001

(54) Process for the preparation of arylamines

(76) Inventors: Fengrui Lang, Nanuet, NY (US); Ioannis Houpis, Danbury, CT (US)

Correspondence Address: MERCK AND CO INC, P O BOX 2000, RAHWAY, NJ, 070650907

(21) Appl. No.: 09/799,441

(22) Filed: Mar. 5, 2001

Related U.S. Application Data
(60) Provisional application No. 60/188,536, filed on Mar. 10, 2000.
(60) Provisional application No. 60/226,529, filed on Aug. 21, 2000.

(51) Int. Cl. C07D 213/02

(52) U.S. Cl. 514/318; 546/193

(57) ABSTRACT

Arylamines are prepared from aryl halides/methanesulfonates using ammonia and copper reagent under mild reaction conditions.

### BACKGROUND OF THE INVENTION

Aminopyridines are found in many compounds of pharmaceutical importance. The synthesis of this class of compounds from its halogen precursors remain a challenging task. Palladium-catalyzed amination of aryl halides using an imine or a t-butylcarbamate as the nitrogen source is an useful method to introduce a nitrogen onto aromatic ring. The reaction features mild reaction conditions and tolerance for a wide range of functional groups; however, this method does not provide a direct way to make primary aryl amines, additional step(s) are necessary to effect the transformation.

Liquid ammonia and KNH₂ have been shown to effect amination from aryl halides. While the reaction appears to give good yield in some cases, the use of liquid ammonia as solvent make the reaction less attractive; furthermore, a number of functional groups do not survive the KNH₂ conditions. Copper bronze or Cu₂O have been shown to be effective in catalyzing the coupling of bromopyridine with amide or sulfonamide type nitrogen with yields ranging from 50 to 70%. Bromopyridine has been reported to couple with secondary amine with Cu₂O catalysis, although the yield is only 20-30%. Aryl halides and amines heated to a high temperature have been reported to provide the corresponding aryl amine products; however, the reactions are usually conducted under forced conditions and the yields are usually low. The direct displacement by ammonia is even harder to achieve. Therefore, there remains a need for an efficient process for the conversion of aryl halides to arylamines that is suitable for large scale industrial production, and which does not require the use of unduly harsh conditions or expensive reagents.

### SUMMARY OF THE INVENTION

The present invention provides an efficient and mild reaction for the conversion of aryl halides/triflates into primary arylamines. The reaction utilizes ammonia and copper reagent and may be used to prepare a wide range of substituted or unsubstituted arylamines.

### DETAILED DESCRIPTION OF THE INVENTION

The present invention provides a process for the preparation of an arylamine of formula I

Ar—NH₂ (I)

which comprises treating a compound of formula (II)

Ar—X (II)

with ammonia in the presence of a copper reagent, in a solvent in which ammonia is soluble, and at a pressure of about 20 to about 200 psi; wherein Ar is optionally substituted aryl; X is Cl, Br, I or trifluoromethanesulfonate.

The term “aryl” includes carbocyclic aromatic ring systems and heterocyclic aromatic ring systems. The aryl group may be substituted or unsubstituted. “Carbocyclic aromatic ring system” includes benzene, naphthalene, benzene fused to a C₅₋₈cycloalkane, C₅₋₈cycloalkene, or a non-aromatic 5- to 8-membered heterocycle having one or two heteroatoms selected from O, S, and N—R¹wherein R¹ is hydrogen, C₁₋₅alkyl, and aryl-C₁₋₅alkyl. Examples of such benzofused ring systems include indoline, indane, indene, tetrahydronaphthalene, 1,2,3,4-tetrahydroquinoline, and 1,2-(methylenedioxy)benzene.

“Heterocyclic aromatic ring systems” means mono- or bicyclic aromatic rings containing from one to three heteroatoms selected from O, S, and N, and optionally fused to a C₅₋₈cycloalkane, C₅₋₈cycloalkene or a non-aromatic 5- to 8-membered heterocycle having one or two heteroatoms selected from O, S, and N—R¹. Examples of heterocyclic aromatic ring systems include pyrrole, imidazole, triazole, oxazole, oxadiazole, thiazole, thiadiazole, thiophene, furan, pyridine, pyrimidine, quinoline, isoquinoline, purine, benzthiazole, benzoxazole, 5,6,7,8-tetrahydroquinoline, and benzothiophene.

The aryl group may be unsubstituted or substituted with up to maximum allowable by valence. The nature of the substituents is not particularly limited, and may be for example alkyl, alkenyl, alkynyl, aryl, cycloalkyl, cycloalkenyl, heterocyclyl, halogen, trifluoromethyl, nitro, cyano, hydroxy, alkoxy, phenoxy, acyl, acyloxy, carbonates, sulfonyloxy, primary, secondary and tertiary amine groups, acylamines, carbamates, sulfonamides, and the like. For Ar—X, X is preferably Br or I, and when X is Cl, Ar is preferably substituted with an electron-withdrawing group.

Examples of Ar—X include, without limitation, 2-, 3-, or 4-bromo-pyridine, 2-bromo-6-picoline, 2-bromo-6-methoxypyridine, 2-bromo-5-nitropyridine, 2-chloro-5-nitropyridine, 2,5-dibromopyridine, 4′-bromoacetophenone, 1-bromo-4-trifluoromethylbenzene, iodobenzene, 2-bromothiazole, 3-bromoquinoline, as well as the compounds having the formula

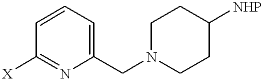

where X is Br and P is t-butoxycarbonyl or acetyl.

Ammonia may be used in any form; for example, a solution of ammonia in a suitable solvent may be pre-made, or ammonia may be directly bubbled into the reaction mixture. The ammonia is typically used in excess relative to Ar—X, for example from 5 to 100 equivalents. The concentration of ammonia may be from about 2 M to about 10 M; preferably from about 4 to about 8 M.

The copper reagent may be any Cu(0), Cu(I) or Cu(II) compounds or complexes, or mixtures thereof. Examples of suitable copper reagent include copper powder, copper bronze, CuCl, CuCl₂, Cu₂O, CuSO₄, mixture of Cu/CuCl. Preferably, the copper reagent is Cu(I) such as Cu₂O and Cu/CuCl. The copper reagent is used in catalytic amount from about 0.5 to about 3 mole percent relative to the Ar—X starting material.

The reaction may be carried out in any solvent in which ammonia is soluble; preferably, the reaction is carried out in an aqueous or alcoholic solvent such as water, methanol, ethanol, isopropanol, ethylene glycol and the like. The reaction is conducted at a temperature ranging from room temperature to about 120° C., and under pressure ranging from about 20 psi to about 200 psi; preferably the reaction is carried out at a temperature of about 50 to about 100° C. and under pressure of from about 40 to about 200 psi. The amination is generally complete within about 15 hours.

The amination reaction of the present invention is applicable to broad range of aryl compounds; in general, heterocyclic aromatic halides and trifluromethanesulfonates are the preferred substrates. Since the reaction is carried out under mild conditions a wide range of substituents can be tolerated; in general, electron withdrawing groups tend to accelerate the reaction.

The reaction of the present invention is preferably carried out in an aqueous or alcoholic solvent such as water or an alcohol; preferably, the reaction is carried out in ethylene glycol. The reaction produces, in addition to the desired arylamine, the aryl-solvent adduct by-product. The by-product is minimized when the reaction is carried out in ethylene glycol, and the amount decreases as the concentration of ammonia is increased. Preferably, the ammonia concentration is from about 4 to about 8 M. The arylamine product may be purified or isolated using conventional chemical techniques well known to those skilled in the art.

In one embodiment of the present process, Ar is optionally substituted heterocyclic aromatic; preferably Ar is selected from optionally substituted pyridine, quinoline and thiazole. In a more preferred embodiment Ar is the group:

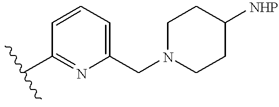

wherein P is an amino protecting group, preferably acetyl or t-butoxycarbonyl; more preferably P is t-butoxycarbonyl.

In another embodiment, X is Br or I.

In another embodiment of the present process, the reaction is carried out in ethylene glycol.

In yet another embodiment, the copper reagent is Cu(I). Preferably, the copper reagent is selected from Cu₂O and Cu/CuCl.

In yet another embodiment, the ammonia is used in a concentration of from about 4 to about 8 M, and the pressure is from about 20 to about 100 psi.

In a preferred embodiment the present invention provides a process for the preparation of a compound of the formula:

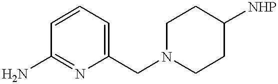

wherein P is an amine protecting group, which comprises treating a compound of the formula:

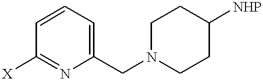

wherein X is Cl, Br, I or OSO₂CF₃ with ammonia in ethylene glycol, in the presence of a copper reagent selected from Cu/CuCl and Cu₂O. More preferably, X is Br, and ammonia is used in a concentration of from about 4 to about 8M.

In another preferred embodiment, the present invention provides a process for the preparation of arylamine of formula I

Ar—NH₂ (I)

which comprises treating a compound of formula II

Ar—X (II)

with ammonia in the presence of from about 0.5 to about 3 mole percent relative to Ar—X a copper reagent selected from Cu₂O and Cu/CuCl, in ethylene glycol, and at a pressure of about 20 to about 200 psi, wherein Ar is optionally substituted aryl, and X is Br or I. More preferably, Ar is optionally substituted phenyl, pyridyl, quinolinyl, or thiazolyl.

The following examples are provided to illustrate the invention and are not to be construed as limiting the invention in any manner.

### EXAMPLE 1

4-tert-butoxycarbonylamino-1-(2-amino-6-pyridylmethyl)piperidine

Method A. Into a 1-liter autoclave was charged Cu (250 mg, 3.93 mmol)/CuCl (250 mg, 2.53 mmol) as a slurry in ethylene glycol, then 4-tert-butoxycarbonylamino-1-(2-bromo-6-pyridylmethyl)piperidine (50 g, 0.135 mole) as a slurry in ethylene glycol. A total volume of 500 mL of ethylene glycol was used. The mixture was cooled to 0° C. and 75 g liquid ammonia was charged over 30 min. with the temperature kept below 10° C. The reaction mixture was heated to 80° C. for 16 h, then cooled to 10° C. and drained into a 3-liter flask for workup. The mixture was adjusted to pH 10.5 with 2 M H₂SO₄ and extracted with 900 mL ethyl acetate. The extract was solvent switched to isopropanol (total volume 900 mL). Then a solution of p-toluenesulfonic acid monohydrate (PTSA, 53.92 g) in 600 mL isopropanol was added over 2 h at room temperature. Precipitate started to form when about half of the PTSA was added. The slurry was aged for 4 h and the solid collected to give 747 g (85%) product as its di-(p-toluenesulfonate) salt.

Method B: A 20-L autoclave was charged with ethylene glycol (10 L, KF=158 ppm) and the vessel was degassed with nitrogen three times with agitation. 4-tert-butoxycarbonylamino-1-(2-bromo-6-pyridylmethyl)piperidine (1007 g, 1000 assay g) and copper(I) oxide (10.51 g) were sequentially charged and nitrogen was bubbled into the suspension with agitation (350 rpm) for 15min. After cooling to 4 C, the vessel was charged with ammonia (2.8 kg) with agitation.

The vessel was heated up to 79° C. by circulating the fluid (80° C.) in the jacket. The pressure reached 8.1 kg/cm² and the mixture turned into a red homogeneous solution from a greenish suspension. After aging for 20 hr, the vessel was cooled to 20° C. by circulating the fluid (13° C.) in the jacket. The solution was degassed at 60° C. with nitrogen bubbling through the reaction mixture. The mixture was vigorously agitated with isopropyl acetate (16.6 L), brine (7%w/v, 10 L) and 5 N aq. NaOH (300 mL) and then settled without agitation for 5 min. The organic layer (18 L) was then separated and the aqueous layer (22 L) was re-extracted with isopropyl acetate (4.2 L). The combined organic layers were washed with brine (15% w/v, 2.5 L) and then concentrated to about half volume in vacuo with azeotropic removal of water. The residual solution was treated with active charcoal (Shirasagi-P, 100 g) at ambient temperature for 1 hr. The mixture was filtered and the filter cake was washed with isopropyl acetate (2 L). The combined filtrate and washings were concentrated in vacuo to 5 L volume at 40° C. The solution was stirred at 30° C. for 1 hr to make some seed bed. n-Heptane (10 L) was added at 30° C. over a period of 30 min and then aged at 15° C. for 1 hr. The resulting slurry was filtered and the cake was washed with isopropyl acetate/n-heptane (1:2.5, 5 L), dried under reduced pressure with sweeping of nitrogen at ambient temperature to give the desired product: (630 g, 74.5 assay % yield) with 97.9 wt %.

### EXAMPLE 2

General Procedure for Copper Catalyzed Amination

A 15 wt % solution of ammonia in ethylene glycol was made by bubbling ammonia gas into ethylene glycol at 0° C., the concentration of ammonia solution was titrated to be around 8 M. The amination reaction was carried out in a sealed tube by mixing 1 g substrate, 10 mL ammonia solution in ethylene glycol and 10 mg Cu₂O. The sealed tube was heated to 80° C. for 16 h. The arylamine is obtained after conventional workup.

The above procedure was repeated to prepare the following arylamines using the indicated starting material and workup procedure:

| Ar-NH₂ | Ar-X | Yield |
|---|---|---|
| 2-amino-5-nitropyridine | 2-chloro-5-nitropyridine | 85% |
| 2-amino-5-nitropyridine | 2-bromo-5-nitropyridine | 99% |
| 3-aminoquinoline | 3-bromoquinoline | 84% |
| 2-aminothiazole | 2-bromothiazole | 94% |
| aniline | iodobenzene | 74% |
| 2-aminopyridine | 2-bromopyridine | 65% |
| 2-amino-6-picoline | 2-bromo-6-picoline | 70% |
| 2-amino-6-methoxypyridine | 2-bromo-6-methoxypyridine | 75% |
| 3-aminopyridine | 3-bromopyridine | 85% |
| 4-aminopyridine | 4-bromopyridine | 81% |
| 4′-aminoacetophenone | 4′-bromoacetophenone | 65% |
| 1-amino-4- | 1-bromo-4-trifluoromethylbenzene | 72% |
| trifluoromethylbenzene | | |
| 2-amino-5-bromopyridine* | 2,5-dibromopyridine | 62% |

*the reaction was carried out at 60° C. for 40 hrs.

What is claimed is:

1. A process for the preparation of an arylamine of formula I
Ar—NH₂ (I)
which comprises treating a compound of formula (II)
Ar—X (II)
with ammonia in the presence of a copper reagent, in a solvent in which ammonia is soluble, and at a pressure of about 20 to about 200 psi; wherein Ar is optionally substituted aryl; X is Cl, Br, I or trifluoromethanesulfonate.

2. A process of claim 1 wherein Ar is optionally substituted heterocyclic aromatic ring system.

3. A process of claim 1 wherein Ar is selected from optionally substituted pyridine, quinoline and thiazole.

4. A process of claim 1 wherein Ar is the group: 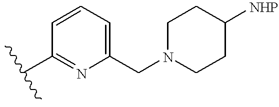
wherein P is acetyl or t-butoxycarbonyl.

5. A process of claim 4 wherein P is t-butoxycarbonyl.

6. A process of claim 1 wherein said process is carried out in an aqueous or alcoholic solvent.

7. A process of claim 1 wherein said process is carried out in ethylene glycol.

8. A process of claim 1 wherein the copper reagent is selected from Cu₂O and Cu/CuCl.

9. A process of claim 1 wherein ammonia is used in a concentration of from about 4 to about 8 M, and the pressure is from about 20 to about 100 psi.

10. A process of claim 1 for the preparation of a compound of the formula: 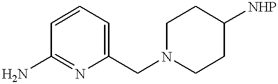
wherein P is acetyl or t-butoxycarbonyl, which comprises treating a compound of the formula: 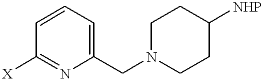
wherein X is Cl, Br, I or OSO₂CF₃ with ammonia in ethylene glycol, in the presence of a copper reagent selected from Cu/CuCl and Cu₂O.

11. A process of claim 10 wherein ammonia is used in a concentration of from about 4 to about 8M.
